(19) United States
(12) Patent Application Publication
(10) Pub. No.: US 2013/0338429 A1
(43) Pub. Date: Dec. 19, 2013

(54) Producing Audio Output for Music Therapy

(71) Applicants: Ronald A. GREGORY, Raleigh, NC (US); John Charles STIER, Olney, MD (US); Paula M. COOLEY, Olney, MD (US)

(72) Inventors: Ronald A. GREGORY, Raleigh, NC (US); John Charles STIER, Olney, MD (US); Paula M. COOLEY, Olney, MD (US)

(73) Assignee: LifeMusic, Inc., Olney, MD (US)

(21) Appl. No.: 13/495,980

(22) Filed: Jun. 13, 2012

(51) Int. Cl. A61M 21/00

(52) U.S. Cl. 600/28

(57) ABSTRACT

A method includes adding, by a computing device, a brain wave pattern having a first frequency to a music track having a first decibel level at or above a threshold of hearing by mixing the brain wave pattern under the music track at a second decibel level less than the first decibel level, wherein the second decibel level is at or below the threshold of hearing, adding, by the computing device, a repetitive chant having a second frequency to the music track, by mixing the repetitive chant under the music track at a third decibel level less than the first decibel level, wherein the third decibel level is at or below the threshold of hearing, and recording, by the computing device, the music track with the brain wave pattern and the repetitive chant to a computer-readable medium.

### BACKGROUND

1. Field

Embodiments described herein relate to the field of audio output for music therapy.

2. Background

Many scientific studies have shown that if a person's subconscious mind is provided with positive thoughts and ideas, then that person's conscious mind will begin to act with information received and positive results may be obtained.

Decades of scientific studies have demonstrated the ability of music to help premature infants gain weight, autistic children communicate, and stroke patients regain speech and mobility. Music has also been shown to be beneficial in controlling chronic pain and as an effective method to reduce anxiety and depression. In addition, there is evidence that music stimulates memories and assists in restoring cognitive function in patients suffering from Alzheimer's disease. Caregivers of Alzheimer's patients are in desperate need of the benefits that music can offer.

### SUMMARY

Embodiments herein describe the use of music, brain waves, and a repetitive chant to provide music therapy.

According to some embodiments, a method includes adding, by a computing device, a brain wave pattern having a first frequency to a music track having a first decibel level at or above a threshold of hearing by mixing the brain wave pattern under the music track at a second decibel level less than the first decibel level, wherein the second decibel level is at or below the threshold of hearing. The method further includes adding, by the computing device, a repetitive chant having a second frequency to the music track, by mixing the repetitive chant under the music track at a third decibel level less than the first decibel level, wherein the third decibel level is at or below the threshold of hearing. In addition, the method includes recording, by the computing device, the music track with the brain wave pattern and the repetitive chant to a computer-readable medium.

### BRIEF DESCRIPTION OF THE DRAWINGS/FIGURES

The accompanying drawings, which are incorporated herein and form a part of the specification, illustrate embodiments of the present invention and, together with the description, further serve to explain the principles of the invention and to enable a person skilled in the pertinent art to make and use the invention.

FIG. 1 illustrates a method of audio creation for use in music therapy according to example embodiments.

FIG. 2 illustrates a flat music track according to example embodiments.

FIGS. 3(a)-3(d) illustrates a plurality of brain waves according to example embodiments.

FIG. 4 illustrates an Aum repetitive chant according to example embodiments.

FIG. 5 illustrates a wavelet transformation of an Aum chant according to example embodiments.

FIG. 6 illustrates an audio recording structure according to example embodiments.

FIG. 7 illustrates a block diagram of a computing device, according to example embodiments.

FIGS. 8-11 illustrate data from a clinical trial related to embodiments of the present invention.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present invention. It will be apparent to a person skilled in the pertinent art that this invention can also be employed in a variety of other applications.

It is noted that references in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

### Introduction

An Alzheimer's disease patient and caregiver often are in desperate need of the benefits that a therapeutic music program can offer. If an Alzheimer patient is in a calm, relaxed state, this may reduce both the time required for actual hands-on care and stress. This may be accomplished by allowing the Alzheimer's patient to enjoy a therapeutic music track. Meanwhile, the caregiver may receive a much-needed respite period.

The effect of a therapeutic music track can be enhanced by adding tracks under the primary music track. These added tracks can be specifically chosen to produce a certain effect in a patient's brain. For example, certain tracks can produce a calming effect in a person's brain.

### Creation of Audio Output

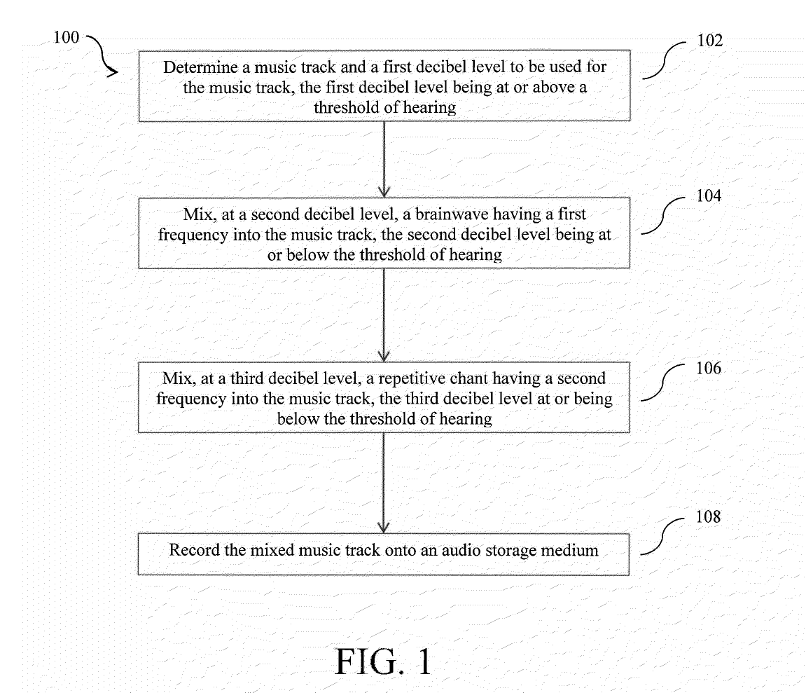

FIG. 1 illustrates a method 100 of creating audio output for a particular person, according to example embodiments. In step 102, age appropriate, generation appropriate, or cultural appropriate music may be determined through in-depth demographic and market research. As another example, appropriate music may be determined by questioning a person's caregiver as to what music the person currently enjoys or enjoyed during his or her youth. Although these examples are provided, other suitable methods may be used for determining appropriate music. The music track may be acquired via creation, purchase or licensing via any conventional channel.

This music may also be described as a “pleasant memories” soundtrack. The subconscious mind stores everything a person experiences in their lifetime, including music. Thus, the subconscious mind retains everything and does not screen information unlike the conscious mind. The subconscious mind does not determine right from wrong or positive from negative. Rather, the subconscious mind stores everything experienced. This subconscious mind can only provide answers based upon information with which it has been programmed and reacts based upon information stored.

People of the same age vary in what music appeals to them and have this knowledge stored in their subconscious mind. A sixty-year-old is just as likely to appreciate Mozart, Frank Sinatra or Led Zeppelin. Thus, it is important to obtain appropriate music that meets a person's preferences.

In another example, a number of different music tracks may be prepared in the manner disclosed herein, such that a selection of pre-prepared tracks is available to a caregiver and/or a patient.

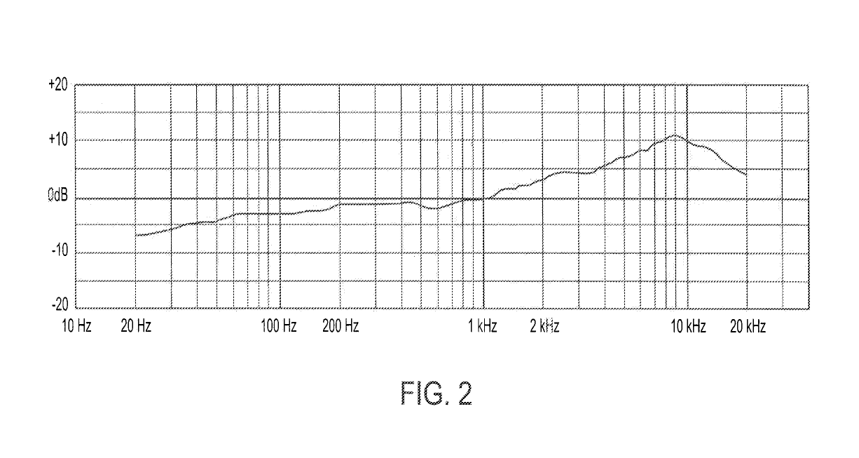

Once the music is chosen, a digital stereo track of the music is obtained by a computing device. In an embodiment, the digital stereo track is a flat track. This flat track does not have any supplemental equalization, but exactly represents the music as it was recorded. An example flat music track is shown in FIG. 2. When a flat track is used, there will be no new frequencies which are presented to the person, so that the music will remain as original as possible. This will minimize any adverse irritating effects that might result from a patient noticing small differences between the tracks they remember and the track they are hearing. The music track is set to a first decibel level that is at or above a threshold of hearing.

Next, after the music is determined, in step 104, a brain wave is mixed under the music by the computing device so that it is not a distraction to the particular person. The brain wave is added at a substantially lower decibel level than the music so that it is at or below the threshold of hearing. For example, the brain wave track may be about 33 dB less than the flat digital stereo track of the music, which is kept at its original decibel level. If the brain wave was fully audible for the particular person, then it may be possible that the brain wave could actually provide an opposite effect, by irritating or further stressing the particular person.

In step 106, after the brain wave is mixed under the music, an Aum reverberating or repetitive chant is also mixed under the music by the computing device so that it is not a distraction to the particular person. Thus, the repetitive chant is added at a substantially lower decibel level than the music so that it is at or below the threshold of hearing. The Aum is discussed below.

Finally, in step 108, the modified music track including the brain wave and the repetitive chant is recorded to a computer-readable medium by the computing device.

Mixing the music track, brain wave, and Aum chant is discussed in further detail below.

### Types of Brain Waves

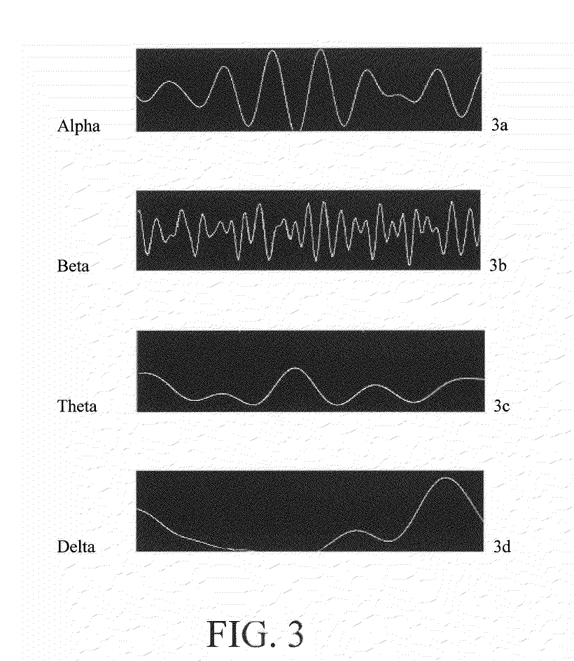

FIG. 3 illustrates four different categories of brain waves that may be recorded to the computer-readable medium. In particular, FIG. 3 shows each of an alpha brain wave, a beta brain wave, a theta brain wave and a delta brain wave. Each of these brain waves produces a specific emotional state for a person. The brain may be trained to respond to stimulation by a specific rhythmical sound or pulse. After being stimulated by a certain frequency, the brain begins to produce the same frequency in its internal rhythm, allowing a person to achieve a targeted state of mind.

Alpha brain waves shown in FIG. 3a are neural oscillations in the frequency range of 8-12 Hz and are produced by the brain when a person is in a state of peace or relaxation. Alpha brain waves were the first brain wave frequency range to be discovered, and are important waves in the brain associated with creativity and clear thinking processes. People who have lower anxiety tend to have significantly more alpha brain wave activity than those who suffer more anxiety. In addition, alpha brain waves are believed to be deficient on the right side of the brain in a large number of individuals, including those who abuse drugs, suffer from depression, suffer from Parkinson's disease, or suffer from Alzheimer's disease. Alpha brain waves are produced when a brain is idle, relaxed, and disengaged, and waiting to respond if necessary. For instance, if a person closes his or her eyes and imagines something peaceful, in less than half a minute, the person will experience an increase in alpha waves. It is believed that a person suffering from Alzheimer's is less likely to indulge in meditation because their brain produces beta and theta waves, but does not produce alpha waves. Thus, such a person is often too agitated to concentrate on peaceful breathing or meditative exercises.

Beta brain waves shown in FIG. 3b are neural oscillations in the frequency range of 14-21 Hz and are produced when a person is aroused, alert and normally conscious. For instance, a person reading this document is likely producing beta brain waves. A person may suffer from a lack of concentration if their brain produces less beta brain waves. Thus, as an example, it may be beneficial for a person suffering from a lack of concentration to listen to beta brain waves.

Theta brain waves shown in FIG. 3c are neural oscillations in the frequency range of 4-7 Hz and are produced when a person is asleep and dreaming, or experiencing “fight or flight.” A person seeking to meditate or resolve fears and phobias may benefit from higher theta brain wave production. Thus, as an example, it may be beneficial for a person seeking to meditate or resolve fears and phobias to listen to theta brain waves.

Delta brain waves shown in FIG. 3d are neural oscillations in the frequency range of 0.5-4 Hz and are produced when a person is experiencing deep, dreamless sleep. A person may suffer from insomnia if their brain produces less delta brain waves. Thus, as an example, it may be beneficial for a person suffering from insomnia to listen to delta brain waves.

Although embodiments used herein refer to therapeutic relief for patients of Alzheimer's disease, other similar techniques may be used for therapeutic relief of other patient conditions, such as insomnia, using appropriate brain wave types and frequencies.

### Aum/Om

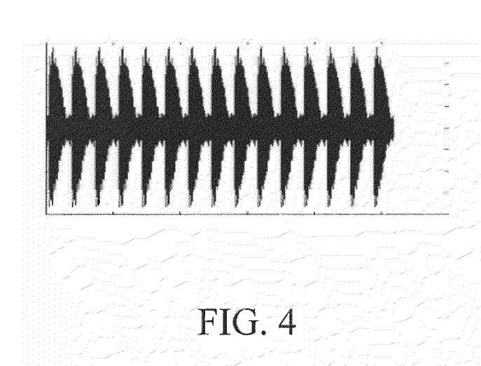

FIG. 4 shows an illustration of an Aum reverberating or repetitive chant. Aum is pronounced in English as “Ohm” and is also known as “Om.” Aum is often referred to as the all-connecting sound of the universe, and “Aum” is one word that is often interpreted as having three sounds which represent creation, preservation and destruction. Aum is described in B. K. S. Iyengar's book entitled Light on Yoga (1995). Aum can be sounded aloud, whispered or repeated mentally.

Iyengar notes that the letter “A” symbolizes the conscious or waking state. He further explains that the letter “U” symbolizes the dream state and the letter “M” symbolizes the dreamless sleep state of the mind and spirit. Aum provides a way of deepening concentration of the mind. The Aum mantra or chant may be sounded aloud, whispered or repeated mentally.

By chanting the vibration Aum, it is thought to be possible to direct it as a healing force to any part of the body, mind, and soul.

In “Time-Frequency Analysis of Chanting Sanskrit Divine Sound ‘Om’ Mantra” published in 2008 by Ajay Anil Gurjar and Siddharth A. Ladhake, it was concluded that

“OM chanting affords steadiness in the mind scientifically. This provides calm and peace to the stressed mind. The mental stress of a person gets reduced while the mind reaches steadiness. As a final point, we have confirmed scientifically the accomplishments of OM chanting in reducing the stress from the human mind.”

According to the study, a professional recording to the chanting Aum was obtained and played in the authors' scientific laboratory. Gurjar and Ladhake performed time-frequency analysis on the Aum mantra chant to analyze the steadiness. By practicing chanting the Aum chant, the mind of stressed people was found to reach steadiness in a few days or weeks.

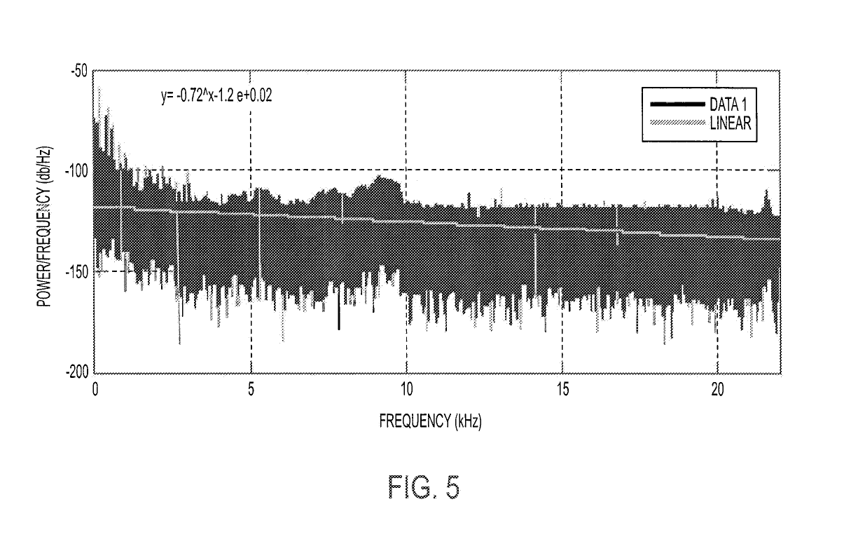

Gurjar and Ladhake noted that the Aum chant may have many variations. It may be very fast and have several cycles a second, or it may be very slow and cycle every several seconds. In addition, it may pulse at an even slower rate. For example, the chant may take the form of (1) “OMmmOMmmOMmm . . . ” (2) “OMmmmmOMmmmm OMmmmm . . . ” or (3) “OMmmmmmmmOMmmmmmmmOMmmmmmmm . . . ” Gurjar and Ladhake performed wavelet transformation on a digital recording of a person chanting the Aum chant after several days of chanting. This is shown in FIG. 5, which shows the attributes of the repetitive chant. This repetitive chant was found to calm the mind, relax the body and sooth and slow the breath of a person. FIG. 5 illustrates that the frequency of the Aum chant naturally became lower which was found to result from allowing the mind to remain awake and alert. The mind and body began to relax and the frequency reduced in tandem.

### Mixing of Tracks

Once the music track, the brain wave track, and the Aum track are obtained, they are mixed into one stereo recording. Typically, mixing is a task that is focused on ensuring that all tracks that are recorded are loudly enough to be heard by a listener. However, according to example embodiments, the non-music tracks are mixed so as not to be loud enough to be consciously heard by a listener. It is desirable that the person listening not be distracted by anything added to the music, while at the same time the person subconsciously hears the brain wave track and the Aum chant track.

Mixing is a process by which the tracks are combined into one or more channels. According to example embodiments, the mixing may be done in an audio studio by a mixing engineer using a sound board, a mixing console and/or a digital audio workstation (DAW). According to example embodiments, a DAW typically includes a computer, an audio interface (such as a sound card), audio editing software and an input device, such as a mouse. The input device may also include other input devices such as a keyboard or a sound board. In addition, mixing of the tracks may be accomplished using a mobile audio workstation (MAW) rather than the DAW.

Examples of audio editing software include, for example and without limitation, Pro Tools, FL Studio, Garageband and Audacity, etc. The DAW is used to process, edit and mix the audio tracks by executing the audio editing software. The DAW may be used to convert the music track, brain wave track, and Aum chant track from analog to digital if necessary. The DAW may also be used to process each of the music track, brain wave track, and Aum chant track by increasing or decreasing decibel level of audio tracks, as well as combine the tracks into one or more channels. However, as noted above, in an embodiment, the music track should represent the music as it is recorded so that the music will remain as original as possible. After the DAW combines the music track, brain wave track, and Aum chant track into one or more channels at appropriate volumes, the DAW outputs and stores a mixed stereo recording as an output track to computer-readable storage, either locally or remotely. The output track may then, for example, be stored by the computer to other computer-readable media or uploaded to the Internet for download by caregivers or patients.

The output track may be stored on any suitable computer-readable medium, such as, for example and without limitation, a compact disc, a USB thumb drive, an SD card or a hard drive. In an embodiment, the computer-readable media, such as the compact disc or the USB thumb drive, is physically distributed to caregivers or patients. In another embodiment, as noted above, the output track is distributed to caregivers or patients via a network such as the Internet, and downloaded by the caregiver or patient and stored on a computer-readable medium. In yet a further embodiment, the output track is streamed via a network such as the Internet. For example, the output track may be stored on a server connected to the Internet and located in a first location. The output track may be distributed via the server to a client, such as a computer also connected to the Internet and located in a second location. The output track may be played back by the caregiver or patient without permanently storing the output track on the client.

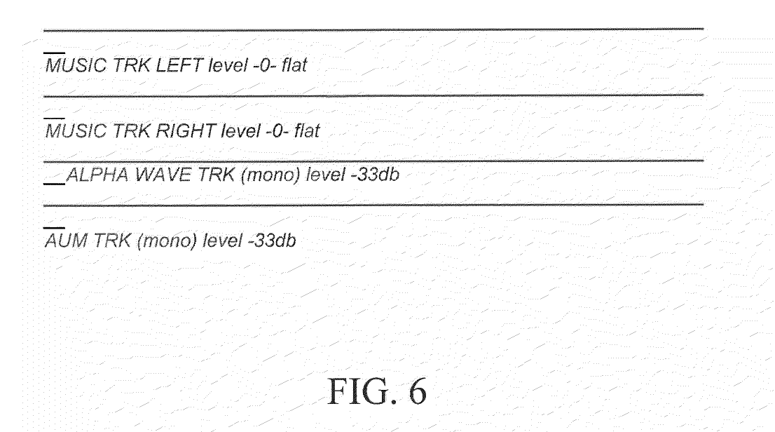

The brain is inspired to produce alpha waves in response to external sources such as music, especially certain rhythmical patterns. The alpha wave patterns may be mixed at or below a threshold of hearing so that they may only be realized by the subconscious mind In addition, the Aum chant track may be mixed at or below a threshold of hearing so that they may only be realized by the subconscious mind. Thus, the brain wave track and the Aum chant track are mixed by the DAW by lowering the decibel level of both the brain wave track and the Aum chant track under the music. An example mixed audio recording structure as created by the DAW is shown in FIG. 6.

### Example

### Clinical Trial

As part of a study by the inventors, Alzheimer's caregivers throughout the United States were recruited nationally to participate in a listening study of output tracks generated by embodiments described above. To be included, each caregiver was required to have primary care responsibilities for a middle or late stage Alzheimer's patient. To qualify, care must have occurred in the home and each patient had to be over 70 years of age. Each caregiver was required to play each of four different music CDs containing the output tracks to their patient. They were also required to play each CD at least twice in a 10 day period, for a total of at least eight hours of music exposure in the 10 days. Upon completion of the 10 day period, each caregiver then took an online survey regarding the impact of the output tracks. The results of the study follow.

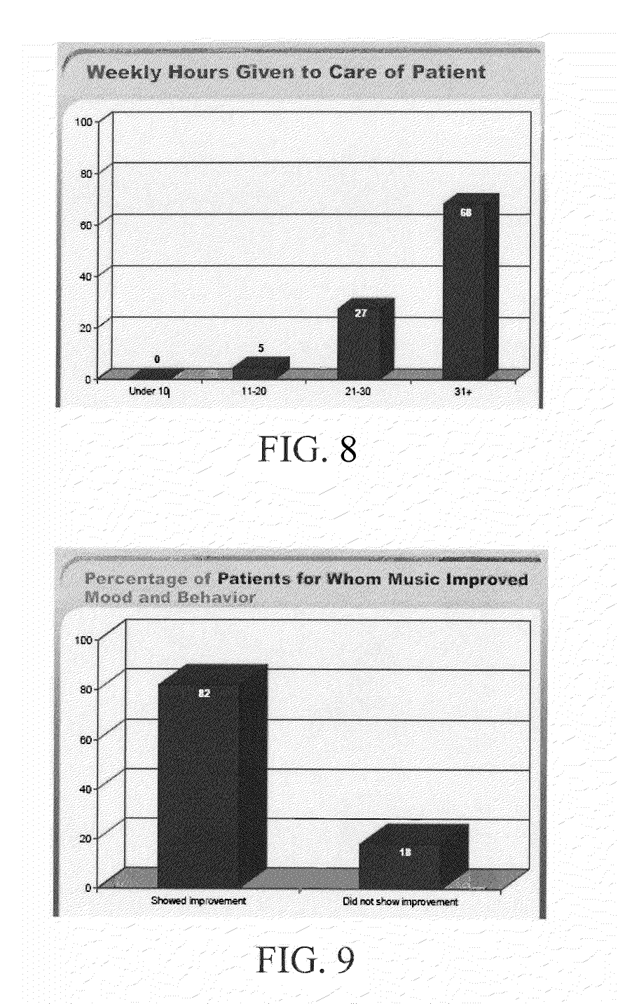

The study indicates that primary caregivers spent a lot of time with their patients and thus knew their behavior and varying moods very well. As shown in FIG. 8, the typical caregiver spent over seven hours a day and over 30 hours a week with their patient. These caregivers acknowledged that their patients were in need of a moderate amount to a lot of care (86%), and few suggested they had patients who were easy to care for.

As shown in FIG. 9, according to the results of the ten day study, the output tracks created according to example embodiments were found to have a positive impact on the mood and the behavior of 82% of patients. Furthermore, the impact appeared to be both quick and long lasting.

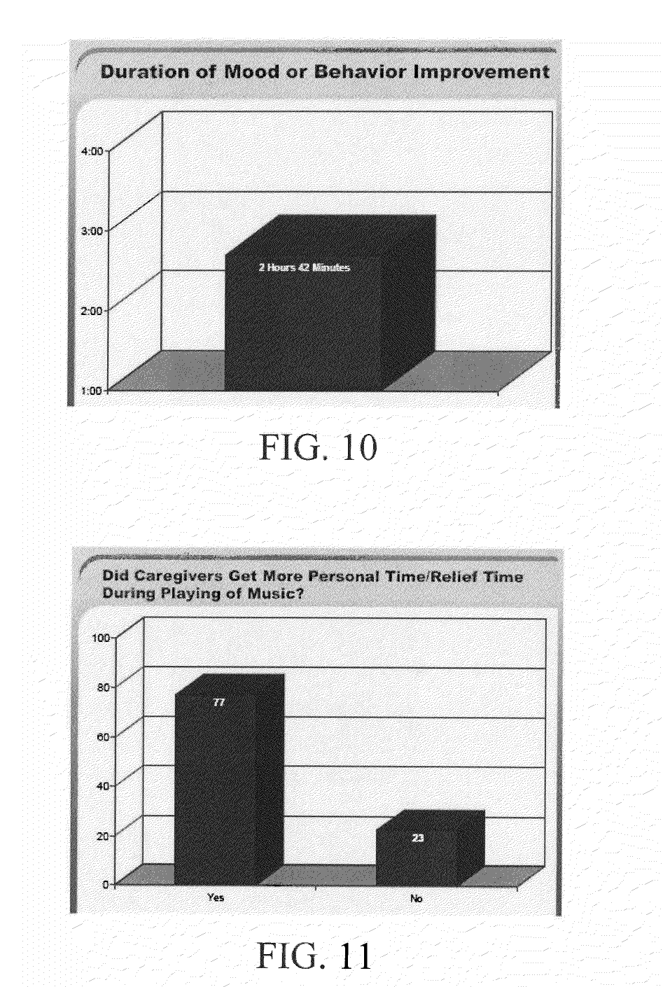

94% of the caregivers who saw a positive response to the output tracks indicated that the positive response normally occurred within thirty minutes or less. Very few indicated that it took longer than thirty minutes for the output tracks to positively impact the patient. Furthermore, the caregivers estimated that the positive reaction to the output tracks lasted an average of two hours and forty-two minutes (shown in FIG. 10). Given that the example copies of the output tracks provided to the caregivers were only one hour in length, this suggests that the output tracks provide a long impact that far exceeds the length of playing the output track.

The data obtained during the trial appears to show that caregivers saw an improvement in the behavior and mood of the patient based on the output tracks. The caregivers found that the output tracks caused the patients to smile, hum along to the music, and discuss old memories. Caregivers also found that the output tracks caused the patients to become happier and quieter.

An additional positive impact from the music tracks was the relief, or “time off,” the sessions provided for the caregiver. As shown in FIG. 11, 77% of the caregivers found that the output tracks not only provided relief and relaxation for the patient, but for the caregiver as well. The caregivers indicated that the patients' mood and behavior improved enough during the sessions that the caregivers were able to find some personal free time and relaxation.

Big Band and Vocal Standards music genres had a more positive impact on mood and behavior of these 70+ Alzheimer's patients than Instrumental Standards or Light Classical, though all types were positively evaluated by the caregivers. Nearly all the caregivers indicated that they would be willing to recommend the music tracks to other caregivers, medical professionals or support groups.

In conclusion, the study suggested that output tracks created according to example embodiments had a positive impact on Alzheimer's patients' mood and behavior. Over a two week period, patients' caregivers attested to this positive impact. The output tracks improved the patients' mood and behavior quickly and over a long period of time. This resulted in a significant percentage of caregivers being able to get a personal break for their own time off and relaxation.

### Example Computing Device

Various aspects of the example embodiments can be implemented by software, firmware, hardware, or a combination thereof In addition, embodiments may be implemented as computer-readable code. Embodiments may be implemented via a set of programs running in parallel on multiple machines. For example, a system 700 carrying out method 100 of FIG. I may be implemented in system 700. Various embodiments of the invention are described in terms of this example system 700.

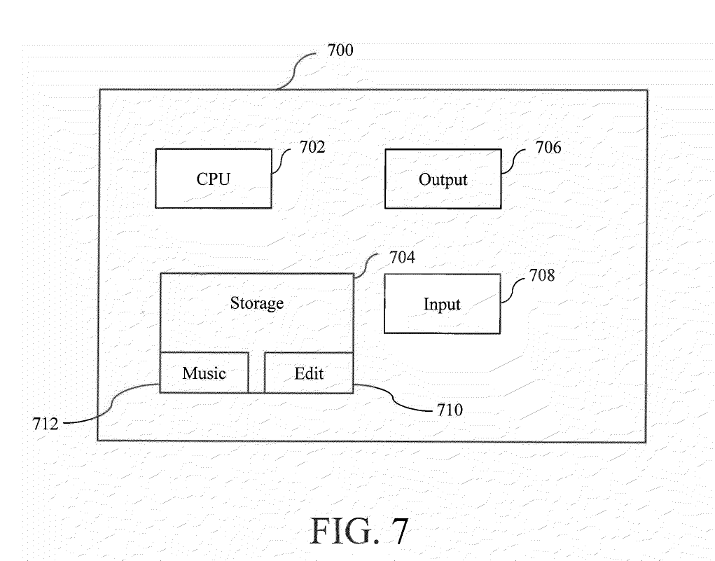

FIG. 7 illustrates a block diagram of a computing device/system 700, according to example embodiments. The computing device may include at least one processing device 702, as well as storage medium 704, which may include main memory such as random access memory (RAM) as well as secondary memory such as a non-transitory storage, e.g. a hard disk drive and/or a removable storage drive, in addition, the secondary memory may include a floppy disk, a magnetic tape, an optical disk, a flash memory, etc. The secondary memory may be written to in a well-known manner As will be appreciated by persons skilled in the relevant art(s), the second memory includes a computer readable storage medium having stored therein computer software and/or data. Processing device 702 is connected to a communication infrastructure, for example, a bus or network (not shown). The computing device may also include an output device 706 and an input device 708.

The computing device may include an audio editing program 710 stored in storage medium 704, which may be used to mix the audio tracks as described above and perform methods to produce audio output 712, according to the example embodiments.

Storage medium 704 may include other similar means for allowing computer programs or other instructions to be loaded into computing device 500. Such means may include, for example, a removable storage unit (not shown) and an interface (not shown). Examples of such means may include a program cartridge and cartridge interface (such as that found in video game devices), a removable memory chip (such as an EPROM or PROM) and associated socket, and other removable storage units and interfaces which allow software and data to be transferred from the removable storage unit to computing device 700.

Computing device 700 may also include a communications interface (not shown). The communications interface allows software and data such as an output music track to be transferred between computing device 700 and external devices. The communications interface may include a modem, a network interface (such as an Ethernet card), a communications port, a PCMCIA slot and card, or the like. Software and data transferred via the communications interface are in the form of signals which may be electronic, electromagnetic, optical, or other signals capable of being received by communications interface. These signals are provided to the communications interface via a communications path (not shown). The communications path carries signals and may be implemented using wire or cable, fiber optics, a phone line, a cellular phone link, an RF link or other communications channels.

In this document, the term “memory” or “storage medium” 704 is used to generally refer to one or more memories or media such as RAM, a hard disk, a removable storage unit, etc. These computer program products are means for providing software to computing device 700.

Computer programs (also called computer control logic) such as audio editing program 710 are stored in main memory and/or secondary memory. Computer programs may also be received via communications interface. Such computer programs, when executed, enable computing device 700 to implement embodiments as discussed herein. In particular, the computer programs, when executed, enable processing device 702 to implement the processes of embodiments of the present invention, such as the steps in the methods discussed above. Accordingly, such computer programs represent controllers of the computing device 700. Where embodiments are implemented using software, the software may be stored in a computer program product and loaded into computing device 700 using storage 704 as described above.

Embodiments may be directed to computer products comprising software stored on any computer readable medium. Such software, when executed in one or more data processing devices, causes the data processing device(s) to operate as described herein.

The summary and abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

What is claimed is:

1. A method, comprising:
adding, by a computing device, a brain wave pattern having a first frequency to a music track having a first decibel level at or above a threshold of hearing by mixing the brain wave pattern under the music track at a second decibel level less than the first decibel level, wherein the second decibel level is at or below the threshold of hearing;
adding, by the computing device, a repetitive chant having a second frequency to the music track, by mixing the repetitive chant under the music track at a third decibel level less than the first decibel level, wherein the third decibel level is at or below the threshold of hearing; and
recording, by the computing device, the music track with the brain wave pattern and the repetitive chant to a computer-readable medium.

2. The method of claim 1, wherein the brain wave pattern is based on at least one of an Alpha brain wave, Beta brain wave, Delta brain wave and Theta brain wave.

3. The method of claim 1, wherein the first frequency is from 8 to 12 Hz.

4. The method of claim 1, wherein the second frequency is 138 Hz.

5. The method of claim 1, further comprising:
playing the music track and producing an audible sound.

6. The method of claim 1, wherein the brain wave pattern is an Alpha brainwave determined to excite neurological activity in a patient diagnosed with Alzheimer's disease.

7. The method of claim 1, further comprising selecting the music track based on preferences of a particular demographic.

8. A system, comprising:
a processor; and
a memory having instructions stored thereon that, when executed by the processor, cause the processor to:
add a brain wave pattern having a first frequency to a music track having a first decibel level at or above a threshold of hearing by mixing the brain wave pattern under the music track at a second decibel level less than the first decibel level, wherein the second decibel level is at or below the threshold of hearing;
add a repetitive chant having a second frequency to the music track, by mixing the repetitive chant under the music track at a third decibel level less than the first decibel level, wherein the third decibel level is at or below the threshold of hearing; and
record the music track with the brain wave pattern and the repetitive chant to a computer-readable medium.

9. The system of claim 8, wherein the brain wave pattern is based on at least one of an Alpha brain wave, Beta brain wave, Delta brain wave and Theta brain wave.

10. The system of claim 8, wherein the first frequency is from 8 to 12 Hz.

11. The system of claim 8, wherein the second frequency is 138 Hz.

12. The system of claim 8, further comprising a speaker,
wherein the memory further has instructions stored thereon that cause the processor to play the music track so as to produce an audible sound using the speaker.

13. The system of claim 8, wherein the brain wave pattern is an Alpha brainwave determined to excite neurological activity in a patient diagnosed with Alzheimer's disease.

14. The system of claim 8, further comprising selecting the music track based on preferences of a particular demographic.

15. An article of manufacture, comprising:
an audio storage medium;
a music track recorded on the audio storage medium and configured to be played at a first decibel level at or above a threshold of hearing;
a brain wave pattern mixed under the music track, the brain wave pattern having a first frequency and configured to be played at a second decibel level that is less than the first decibel level, wherein the second decibel level is at or below the threshold of hearing; and
a repetitive chant mixed under the music track, the repetitive chant having a second frequency and configured to be played at a third decibel level that is less than the first decibel level, wherein the third decibel level is at or below the threshold of hearing,
wherein the music track, the brain wave pattern, and the repetitive chant are all stored on the audio storage medium such that they provide simultaneous output when played.

16. The article of manufacture of claim 15, wherein the brain wave pattern is based on at least one of an Alpha brain wave, Beta brain wave, Delta brain wave, and Theta brain wave.

17. The article of manufacture of claim 15, wherein the first frequency is in the range from 8 to 12 Hz.

18. The article of manufacture of claim 15, wherein the second frequency is 138 Hz.

19. The article of manufacture of claim 15, wherein the brain wave pattern is an Alpha brainwave determined to excite neurological activity in a patient diagnosed with Alzheimer's disease.

20. The article of manufacture of claim 15, wherein the music track is based on preferences of a particular demographic.
